(19) United States
(12) Patent Application Publication
(10) Pub. No.: US 2016/0120871 A1
(43) Pub. Date: May 5, 2016

(54) PHARMACEUTICAL COMBINATIONS OF A PI3K INHIBITOR AND A MICROTUBULE DESTABILIZING AGENT

(71) Applicants: Alejandro PIRIS GIMÉNEZ, Barcelona (ES); Javier CORTÉS CASTÁN, Barcelona (ES); Violeta SERRA ELIZALDE, Barcelona (ES)

(72) Inventors: Javier Cortés Castán, Barcelona (ES); Alejandro Piris Giménez, Barcelona, (ES); Violeta Serra Elizalde, Barcelona (ES)

(73) Assignee: NOVARTIS AG, Basel (CH)

(21) Appl. No.: 14/895,111

(22) PCT Filed: Jun. 10, 2014
(86) PCT No.: PCT/IB2014/062095

§ 371 Date: Dec. 1, 2015

Related U.S. Application Data
(60) Provisional application No. 61/833,503, filed on Jun. 11, 2013.

(51) Int. Cl. A61K 31/5377; A61K 31/4439; A61K 31/357

(52) U.S. Cl. CPC A61K 31/5377; A61K 31/357; A61K 31/4439

(57) ABSTRACT

The present invention relates to a combination comprising (a) a phosphatidylinositol 3-kinase inhibitor selected from 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salts thereof, and (b) a microtubule destabilizing agent for simultaneous, separate or sequential use for the treatment of a tumor disease; a pharmaceutical composition comprising such combination; a method of treating a subject having a tumor disease comprising administration of said combination to a subject in need thereof; use of such combination for preparation of a medicament for the treatment of a tumor disease; and a commercial package thereto.

### FIELD OF THE INVENTION

The present invention relates to a combination comprising (a) a phosphatidylinositol 3-kinase inhibitor selected from 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salt thereof, and (b) a microtubule destabilizing agent for simultaneous, separate or sequential use for the treatment of a tumor disease; a pharmaceutical composition comprising such combination; a method of treating a subject having a tumor disease comprising administration of said combination to a subject in need thereof; use of such combination for preparation of a medicament for the treatment of a tumor disease; and a commercial package thereto.

### BACKGROUND

Epidemiological and experimental studies support an important role for phosphatidylinositol 3-kinases (PI3Ks) in the biology of human cancer. Phosphatidylinositol 3-kinases (PI-3 kinase or PI3K) comprise a family of lipid and serine/threonine kinases that catalyze the transfer of phosphate to the D-3′ position of inositol lipids to produce phosphoinositol-3-phosphate (PIP), phosphoinositol-3,4-diphosphate (PIP2) and phosphoinositol-3,4,5-triphosphate (PIP3) that, in turn, act as second messengers in signaling cascades by docking proteins containing pleckstrin-homology, FYVE, Phox and other phospholipid-binding domains into a variety of signaling complexes often at the plasma membrane ((Vanhaesebroeck et al., Annu. Rev. Biochem 70:535 (2001); Katso et al., Annu. Rev. Cell Dev. Biol. 17:615 (2001)). Of the two Class 1 PI3Ks, Class 1A PI3Ks are heterodimers composed of a catalytic p110 subunit (α, β, δ isoforms) constitutively associated with a regulatory subunit that can be p85α, p55α, p50α, p85β or p55γ. The Class 1B sub-class has one family member, a heterodimer composed of a catalytic p110γ subunit associated with one of two regulatory subunits, p101 or p84 (Fruman et al., Annu Rev. Biochem. 67:481 (1998); Suire et al., Curr. Biol. 15:566 (2005)). The modular domains of the p85/55/50 subunits include Src Homology (SH2) domains that bind phosphotyrosine residues in a specific sequence context on activated receptor and cytoplasmic tyrosine kinases, resulting in activation and localization of Class 1A PI3Ks. Class 1B PI3K is activated directly by G protein-coupled receptors that bind a diverse repertoire of peptide and non-peptide ligands (Stephens et al., Cell 89:105 (1997)); Katso et al., Annu. Rev. Cell Dev. Biol. 17:615-675 (2001)). Consequently, the resultant phospholipid products of class I PI3K link upstream receptors with downstream cellular activities including proliferation, survival, chemotaxis, cellular trafficking, motility, metabolism, inflammatory and allergic responses, transcription and translation (Cantley et al., Cell 64:281 (1991); Escobedo and Williams, Nature 335:85 (1988); Fantl et al., Cell 69:413 (1992)).

Aberrant regulation of PI3K is one of the most prevalent events in human cancer. In some tumors, the genes for the p110α isoform, PIK3CA, and for Akt are amplified and increased protein expression of their gene products has been demonstrated in several human cancers. Further, somatic missense mutations in PIK3CA that activate downstream signaling pathways have been described at significant frequencies in a wide diversity of human cancers (Kang et al., Proc. Natl. Acad. Sci. USA 102:802 (2005); Samuels et al., Science 304:554 (2004); Samuels et al., Cancer Cell 7:561-573(2005)).

Breast cancer is the most commn form of malignant tumor in women worldwide, and the incident rates are as high as 99.4 per 100,000 women. Gain of function mutations in PIK3CA have been observed in about 10% to 40% of breast cancer patients. Inactivation of the tumor suppressor gene PTEN also leads to PI3K pathway activation and has been reported in 15% to 48% of breast cancer patients. In spite of numerous treatment options for patients suffering from tumor diseases, there remains a need for effective and safe therapeutic agents and a need for their preferential use in combination therapy.

It is now found that the combination comprising a phosphatidylinositol 3-kinase inhibitor selected from 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salt thereof, and specific microtubule destabilizing agents, particularly eribulin or a pharmaceutically acceptable salt thereof, has improved antitumor activity as compared to each monotherapy and may be effective for the treatment of tumor disease, particularly breast cancer. This beneficial interaction is expected to allow reduction in the dose required for each therapeutic agents, leading to a reduction in the side effects and enhancement of the long-term clinical effectively of the therapeutic agents in treatment.

### SUMMARY OF THE INVENTION

The present invention pertains to a combination comprising (a) a phosphatidylinositol 3-kinase (PI3K) inhibitor selected from 5-(2,6-di-morpholin-4-yl-pyrimidin-4-yl)-4-trifluoromethyl-pyridin-2-ylamine (“COMPOUND A”), (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}amide) (“COMPOUND B”) or a pharmaceutically acceptable salts thereof, and (b) a microtubule destabilizing agent selected from eribulin, vinorelbine, vindesine, vincristine, vinblastine, vinflunine, ABT-751, verubulin, lexibulin, denibulin, indibulin, combrestatin A4, combrestatin A1, AVE8062 or a pharmaceutically acceptable salt thereof, for simultaneous, separate or sequential use for the treatment of a tumor disease, particularly breast cancer.

In one preferred embodiment of the present invention, the combination comprises (a) a phosphatidylinositol 3-kinase inhibitor COMPOUND A or a pharmaceutically acceptable salt thereof, and (b) eribulin or a pharmaceutically acceptable salt thereof, particularly eribulin mesylate.

In one preferred embodiment of the present invention, the combination comprises (a) a phosphatidylinositol 3-kinase inhibitor COMPOUND B or a pharmaceutically acceptable salt thereof, and (b) eribulin or a pharmaceutically acceptable salt thereof, particularly eribulin mesylate.

In one aspect, the invention provides a pharmaceutical composition comprising a quantity of the COMBINATION OF THE INVENTION which is jointly therapeutically effective against a tumor disease.

In one aspect, the present invention provides a method of treating a tumor disease comprising administering to subject in need thereof a COMBINATION OF THE INVENTION in a quantity, which is jointly therapeutically effective against said tumor disease. In one embodiment, the tumor disease to be treated with a COMBINATION OF THE INVENTION is breast cancer, preferably breast cancer resistant to at least one prior chemotherapeutic regimen.

In one aspect, the present invention also provides a method of inhibiting the formation of metastases in a subject having tumor disease, in particular a breast cancer, comprising administering to a subject in need thereof an amount of a COMBINATION OF THE INVENTION in a quantity which is therapeutically effective against said tumor disease.

In one aspect, the present invention provides the use of a COMBINATION OF THE INVENTION for the treatment of a tumor disease, in particular breast cancer, and for the preparation of a medicament for the treatment of a tumor disease.

In one aspect, the present invention provides the use of a COMBINATION OF THE INVENTION for the inhibition of the formation of metastases in a subject having a tumor disease.

In one aspect, the present invention provides a commercial package comprising as active ingredients COMBINATION OF THE INVENTION, together with instructions for the simultaneous, separate or sequential use thereof in the treatment of a tumor disease, particularly breast cancer.

In one aspect, the present invention provides a commercial package comprising a phosphatidylinositol 3-kinase (PI3K) inhibitor selected from 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salt thereof and instructions for the simultaneous, separate or sequential use with a microtubule destabilizing agent selected from eribulin, vinorelbine, vindesine, vincristine, vinblastine, vinflunine, ABT-751, verubulin, lexibulin, denibulin, indibulin, combrestatin A4, combrestatin A1, AVE8062 or a pharmaceutically acceptable salt thereof, in the treatment of a tumor disease.

### BRIEF DESCRIPTION OF THE DRAWINGS

FIG. 1 shows the percentage of AnnexinV-positive (apoptotic) HCC1143 triple negative breast cancer cells (PTEN normal) after treatment with either the combination of COMPOUND A hydrochloride salt with eribulin mesylate, control, COMPOUND A hydrochloride salt monotherapy or eribulin mesylate monotherapy. Data shows the results for treatments at EC50 and 48 hours.

FIG. 2 shows the percentage of AnnexinV-positive (apoptotic) MDA-MB-468 triple-negative breast cancer cells (characterized with PTEN loss) after treatment with either the combination of COMPOUND A hydrochloride salt with eribulin mesylate, control, COMPOUND A hydrochloride salt monotherapy or eribulin mesylate monotherapy. Data shows the results for treatments at EC50 and 48 hours.

FIG. 3 shows the final tumor volume of MDA-MB-468 triple-negative breast cancer cells (characterized with PTEN loss) in xenograft models after administration of the combination of COMPOUND A hydrochloride salt with eribulin mesylate as compared to control, COMPOUND A hydrochloride salt monotherapy and eribulin mesylate monotherapy.

FIG. 4 shows the antitumor activity of the combination of COMPOUND A hydrochloride salt with eribulin mesylate in a triple-negative breast cancer patient-derived xenograft model (PDX44) characterized with a PIK3CA H1047R mutation and PTEN loss as compared to control, COMPOUND A hydrochloride salt monotherapy and eribulin mesylate monotherapy.

FIG. 5 shows the antitumor activity of the combination of COMPOUND A hydrochloride salt with eribulin mesylate against CAL51 triple-negative breast cancer cells (characterized with a PIK3CA E542K mutation and PTEN loss) in xenograft models as compared to control, COMPOUND A hydrochloride salt monotherapy and eribulin mesylate monotherapy.

FIG. 6 shows the antitumor activity of the combination of COMPOUND B with eribulin mesylate in a triple-negative breast cancer patient-derived xenograft model (PDX44) characterized with a PIK3CA H1047R mutation and PTEN loss as compared to control, Compound B monotherapy and eribulin mesylate monotherapy.

### DETAILED DESCRIPTION

The present invention pertains to a combination comprising (a) a phosphatidylinositol 3-kinase (PI3K) inhibitor selected from 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salt thereof, and (b) a microtubule destabilizing agent selected from eribulin, vinorelbine, vindesine, vincristine, vinblastine, vinflunine, ABT-751, verubulin, lexibulin, denibulin, indibulin, combrestatin A4, combrestatin A1, AVE8062 or a pharmaceutically acceptable salt thereof, for simultaneous, separate or sequential use for the treatment of a tumor disease, particularly breast cancer.

The general terms used herein are defined with the following meanings, unless explicitly stated otherwise:

The terms “comprising” and “including” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “combination” or “pharmaceutical combination” is defined herein to refer to either a fixed combination in one dosage unit form, a non-fixed combination or a kit of parts for the combined administration where the phosphatidylinositol 3-kinase inhibitor and the a microtubule destabilizing agent or pharmaceutically acceptable salt thereof may be administered independently at the same time or separately within time intervals that allow that the combination partners show a cooperative, e.g., synergistic, effect.

The term “fixed combination” means that the active ingredients or therapeutic agents, e.g. the phosphatidylinositol 3-kinase inhibitor and the microtubule destabilizing agent, are administered to a patient simultaneously in the form of a single entity or dosage form.

The term “non-fixed combination” means that the active ingredients or therapeutic agents, e.g. the phosphatidylinositol 3-kinase inhibitor and the microtubule destabilizing agent, are both administered to a patient as separate entities or dosage forms either simultaneously, concurrently or sequentially with no specific time limits, wherein such administration provides therapeutically effective levels of the three compounds in the body of the subject, e.g., a mammal or human, in need thereof.

The term “a phosphatidylinositol 3-kinase inhibitor” or “PI3K inhibitor” is defined herein to refer to a compound which targets, decreases or inhibits phosphatidylinositol 3-kinase. Phosphatidylinositol 3-kinase activity has been shown to increase in response to a number of hormonal and growth factor stimuli, including insulin, platelet-derived growth factor, insulin-like growth factor, epidermal growth factor, colony-stimulating factor, and hepatocyte growth factor, and has been implicated in processes related to cellular growth and transformation.

The term “microtubule destabilizing agent” is defined herein to refer to any compound or biologic agent that directly or indirectly interacts with microtubules and inhibits microtubule polymerization, and thus, interferes with the physiological function of microtubules. Such agents are contrasted with compounds or biologic agents which stabilize microtubule polymers and inhibit microtubule depolymerization (ie., microtubule stabilizing agents).

The term “pharmaceutical composition” is defined herein to refer to a mixture or solution containing at least one therapeutic agent to be administered to a subject, e.g., a mammal or human, in order to treat a particular disease or condition affecting the subject thereof.

The term “pharmaceutically acceptable” is defined herein to refer to those compounds, biologic agents, materials, compositions and/or dosage forms, which are, within the scope of sound medical judgment, suitable for contact with the tissues a subject, e.g., a mammal or human, without excessive toxicity, irritation allergic response and other problem complications commensurate with a reasonable benefit/risk ratio.

The terms “combined administration” as used herein are defined to encompass the administration of the selected therapeutic agents to a single subject, e.g., a mammal or human, and are intended to include treatment regimens in which the agents are not necessarily administered by the same route of administration or at the same time.

The term “treating” or “treatment” as used herein comprises a treatment relieving, reducing or alleviating at least one symptom in a subject or effecting a delay of progression of a disease. For example, treatment can be the diminishment of one or several symptoms of a disorder or complete eradication of a disorder, such as cancer. Within the meaning of the present invention, the term “treat” also denotes to arrest, delay the onset (i.e., the period prior to clinical manifestation of a disease) and/or reduce the risk of developing or worsening a disease.

The term “jointly therapeutically active” or “joint therapeutic effect” as used herein means that the therapeutic agents may be given separately (in a chronologically staggered manner, especially a sequence-specific manner) in such time intervals that they prefer, in the warm-blooded animal, especially human, to be treated, still show a (preferably synergistic) interaction (joint therapeutic effect). Whether this is the case can, inter alia, be determined by following the blood levels, showing that both therapeutic agents are present in the blood of the human to be treated at least during certain time intervals.

The term “pharmaceutically effective amount” or “therapeutically effective amount” of a combination of therapeutic agents is an amount sufficient to provide an observable improvement over the baseline clinically observable signs and symptoms of the tumor disease treated with the combination.

The term “synergistic effect” as used herein refers to action of two agents such as, for example, (a) a phosphatidylinositol 3-kinase (PI3K) inhibitor selected from 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salts thereof, and (b) a microtubule destabilizing agent, e.g., eribulin or a pharmaceutically acceptable salt thereof, producing an effect, for example, slowing the symptomatic progression of a proliferative disease, particularly cancer, or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

The term “subject” or “patient” as used herein includes animals, which are capable of suffering from or afflicted with a tumor disease or any disorder involving, directly or indirectly, a tumor. Examples of subjects include mammals, e.g., humans, dogs, cows, horses, pigs, sheep, goats, cats, mice, rabbits, rats and transgenic non-human animals. In the preferred embodiment, the subject is a human, e.g., a human suffering from, at risk of suffering from, or potentially capable of suffering from a tumor disease.

The term about” or “approximately” shall have the meaning of within 10%, more preferably within 5%, of a given value or range.

Combinations of the present invention include a phosphatidylinositol 3-kinase inhibitor (PI3K) selected from the compound of formula (I), compound of formula (II) or any pharmaceutically acceptable salt thereof.

WO07/084786 describes specific pyrimidine derivatives which have been found to inhibit the activity of PI3K. One PI3K inhibitor suitable for the present invention is the compound 5-(2,6-di-morpholin-4-yl-pyrimidin-4-yl)-4-trifluoromethyl-pyridin-2-ylamine (hereinafter also referred to as “COMPOUND A”) having the chemical structure of formula (I)

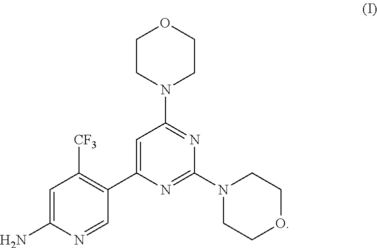 [SMILES: Nc1cc(C(F)(F)F)c(-c2cc(N3CCOCC3)nc(N3CCOCC3)n2)cn1]

The compound, its salts, its utility as a PI3K inhibitor and synthesis of the compound 5-(2,6-di-morpholin-4-yl-pyrimidin-4-yl)-4-trifluoromethyl-pyridin-2-ylamine are described in WO 2007/084786, which is hereby incorporated by reference in its entirety hereto, for instance as Example 10.

The compound of formula (I) may be present in the combination in the form of the free base or a pharmaceutically acceptable salt thereof. Such salts can be prepared in situ during the final isolation and purification of the compounds, or by separately reacting the base or acid functions with a suitable organic or inorganic acid or base, respectively. Suitable salts of the compound of formula (I) include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemi-sulfate, heptanoate, hexanoate, fumarate, hydrochloride, hydrobromide, hydroiodide, 2 hydroxyethanesulfonate, lactate, maleate, methanesulfonate, nicotinate, 2 naphth-alenesulfonate, oxalate, pamoate, pectinate, persulfate, 3 phenylproionate, picrate, pivalate, propionate, succinate, sulfate, tartrate, thiocyanate, p toluenesulfonate, and undecanoate. Also, the basic nitrogen-containing groups can be quaternized with such agents as alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl, and stearyl chlorides, bromides and iodides, aralkyl halides like benzyl and phenethyl bromides, and others. In a preferred embodiment, the compound of formula (I) is in the form of its hydrochloride salt.

Further, WO 2010/029082 describes specific 2-carboxamide cycloamino urea derivatives which have been found to selectively inhibit the activity of the alpha-isoform of PI3K. Another PI3K inhibitor suitable for the present invention is the compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (hereinafter also referred to as “COMPOUND B”) having the chemical structure of formula (II)

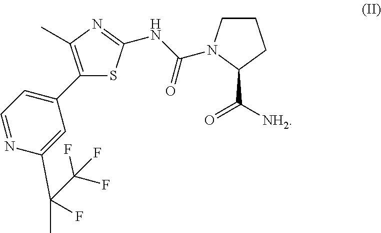 [SMILES: Cc1nc(NC(=O)N2CCC[C@H]2C(N)=O)sc1-c1ccnc(C(C)(F)C(F)(F)F)c1]

The compound, its salts, its utility as an alpha-isoform specific PI3K inhibitor and synthesis of the compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) are described in WO 2010/029082, which is hereby incorporated by reference in its entirety hereto, for instance as Example 15.

The compound of formula (II) may be incorporated in the combination of the present invention in either the form of its free base or any pharmaceutically acceptable salt thereof. Such salts of the compound of formula (II) are formed, for example, as acid addition salts, preferably with organic or inorganic acids, from compound of formula (II) with a basic nitrogen atom. Suitable inorganic acids are, for example, halogen acids, such as hydrochloric acid, sulfuric acid, or phosphoric acid. Suitable organic acids are, e.g., carboxylic acids or sulfonic acids, such as fumaric acid or methansulfonic acid.

Combinations of the present invention further include a a microtubule destabilizing agent selected from eribulin (Halaven®, Eisai Inc.), vinorelbine (Navelbine®, Pierre Fabre), vindesine (Eldisine®), vincristine, vinblastine, vinflunine (Javlor®, Pierre Fabre Medicament), ABT-751 (Abbott); verubulin hydrochloride (Azixa™, Myriad Pharmaceuticals), lexibulin hydrochloride (YM Biosciences Australia), denibulin (MediciNova/Angiogene), indibulin (Zybulin™, Ziopharm Oncology), combrestatin A4 (Zybrestat™, Oxigene), combrestatin A1 (Oxi4053, Oxigene), AVE8062 (Sanofi-Aventis) or any pharmaceutically acceptable salt thereof.

Eribulin (also known as E73889) is a structural ananalogue of halichondrin B, a national product originally isolated from the rare marine sponge Halichondria okadai. Eribulin has the chemical name 11,15:18,21:24,28Triepoxy-7,9-ethano-12,15-methano-9H,15H-furo[3,2-i]furo[2′,3′:5,6]pyrano[4,3b][1,4]dioxacyclopentacosin-5(4H)-one, 2-[(2S)-3-amino-2-hydroxypropyl]hexacosahydro-3methoxy-26-methyl-20,27-bis(methylene)-, (2R,3R,3aS,7R,8aS,9S,10aR,11S,12R,13aR,13bS,15S,18S,21S,24S,26R,28R,29aS) and the chemical structure of formula (III)

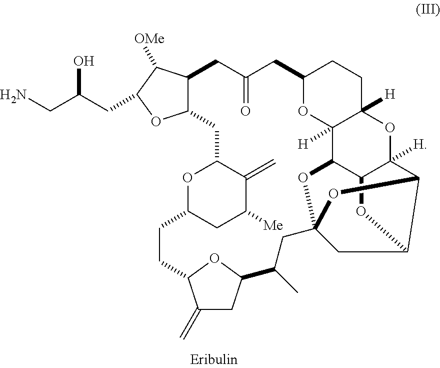

Eribulin has a molecular weight of 729.9 in the free base form and 826.0 in the mesylate salt form. Eribulin mesylate (commercially available by Eisai under the tradename Halaven®) is currently indicated for the treatment of patients with metastatic breast cancer who have previously received at least two chemotherapeutic regimens for the treatment of metastatic disease. Prior therapy should include an anthracycline and a taxane in either the adjuvant or metastatic setting.

Other microtubule destabilizing agents suitable for the combination of the present invention include vinorelbine (Navelbine®, Pierre Fabre), vindesine (Eldisine®), vincristine, vinblastine, vinflunine (Javlor®, Pierre Fabre Medicament), ABT-751 (Abbott); verubulin hydrochloride (Azixa™, Myriad Pharmaceuticals), lexibulin hydrochloride (YM Biosciences Australia), denibulin (MediciNova/Angiogene), indibulin (Zybulin™, Ziopharm Oncology), combrestatin A4 (Zybrestat™, Oxigene), combrestatin A1 (Oxi4053, Oxigene), AVE8062 (Sanofi-Aventis) or any pharmaceutically acceptable salt thereof.

Preferably, the microtubule destabilizing agent is eribulin or its pharmaceutically acceptable salt, particularly eribulin mesylate.

The structure of the active ingredients identified by code numbers., generic or trade names may be taken from the actual edition of the standard compendium “The Merck Index” or from databases, e.g., Patents International (e.g, IMS World Publications). The corresponding content thereof is hereby incorporated by reference.

A combination comprising (a) a phosphatidylinositol 3-kinase (PI3K) inhibitor selected from 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salt thereof, and (b) a microtubule destabilizing agent selected from eribulin, vinorelbine, vindesine, vincristine, vinblastine, vinflunine, ABT-751, verubulin, lexibulin, denibulin, indibulin, combrestatin A4, combrestatin A1, AVE8062 or a pharmaceutically acceptable salt thereof, will be referred to hereinafter as a COMBINATION OF THE INVENTION.

In one preferred embodiment of the present invention, the combination comprises the phosphatidylinositol 3-kinase inhibitor COMPOUND A or its hydrochloride salt

In one preferred embodiment of the present invention the combination comprises the phosphatidylinositol 3-kinase inhibitor COMPOUND B or a pharmaceutically acceptable salt thereof.

In one preferred embodiment of the present invention, the combination comprises (a) a phosphatidylinositol 3-kinase inhibitor selected from COMPOUND A, COMPOUND B or a pharmaceutically acceptable salt thereof, and (b) eribulin or a pharmaceutically acceptable salt thereof, particularly eribulin mesylate.

In one preferred embodiment of the present invention, the combination comprises (a) a phosphatidylinositol 3-kinase inhibitor COMPOUND A or a pharmaceutically acceptable salt thereof, and (b) eribulin or a pharmaceutically acceptable salt thereof, particularly eribulin mesylate.

In one preferred embodiment of the present invention, the combination comprises (a) a phosphatidylinositol 3-kinase inhibitor COMPOUND B or a pharmaceutically acceptable salt thereof, and (b) eribulin or a pharmaceutically acceptable salt thereof, particularly eribulin mesylate.

The nature of tumor diseases like tumor diseases is multifactorial. Under certain circumstances, drugs with different mechanisms of action may be combined. However, just considering any combination of drugs having different mode of action does not necessarily lead to combinations with advantageous effects.

It has been found that the administration of the COMBINATION OF THE INVENTION has improved antitumor activity as compared to each monotherapy and may be effective for the treatment of tumor disease, particularly breast cancer. In the present invention, the administration of the COMBINATION OF THE INVENTION is expected to result in a more beneficial treatment, e.g, synergistic or improved anti-proliferative effect, e.g., with regard to the delay of progression of tumor disease or with regard to a change in tumor volume, as compared to either monotherapy.

The COMBINATION OF THE INVENTION is particularly useful for the treatment of a tumor disease, such as cancer, and/or for the inhibition of the formation of metastases in a subject having a tumor disease. In the case of metastases, the tumor disease is identified by the location or origin of the primary tumor.

Examples of tumor diseases suitable for treatment with the COMBINATION OF THE INVENTION include, but not limited to, benign or malignant tumors, brain cancer, kidney cancer, liver cancer, bladder cancer, breast cancer, gastric cancer, ovarian cancer, colon cancer, rectum cancer, prostate cancer, pancreatic cancer, lung cancer (including non-small cell lung cancer and small cell lung cancer), bronchial cancer, vaginal cancer, hepatocellular carcinoma, intrahepatic bile duct cancer, glioma, glioblastoma, cervical cancer, bladder cancer, esophageal cancer, cancer of the head and neck, thyroid cancer, melanoma, endometrial cancer, multiple myeloma, acute myelogeous leukemia, chronic myelogenous leukemia, lymphocytic leukemia, non-Hodgkin lymphoma, gastrointestinal cancer, or any combination thereof.

The combinations disclosed herein inhibit the growth of solid tumors, but also liquid tumors. The term “solid tumor” especially means breast cancer, ovarian cancer, colon cancer, rectum cancer, gastric cancer, cervical cancer, lung cancer (including non-small cell lung cancer and small cell lung cancer), head and neck cancer, bladder cancer, and prostate cancer. Further, depending on the tumor type and the particular combination used, a decrease of the tumor volume can be obtained. The combinations disclosed herein are also suited to prevent the metastatic spread of tumors and the growth or development of micrometastases.

In one embodiment, the tumor disease is a solid tumor. In a further embodiment, the tumor disease is a solid tumor resistant to at least one prior chemotherapeutic regimen.

In a preferred embodiment, the tumor disease treated is breast cancer.

In another preferred embodiment, the tumor disease treated is triple negative breast cancer.

In another preferred embodiment, the tumor disease treated is a metastatic breast cancer resistant to at least one prior chemotherapeutic regimen.

In accordance with the present invention, a patient having a tumor disease, particularly breast cancer, may be separately, simultaneously or sequentially administered (a) a phosphatidylinositol 3-kinase (PI3K) inhibitor selected from COMPOUND A, COMPOUND B or a pharmaceutically acceptable salt thereof, and (b) a microtubule destabilizing agent selected from eribulin, vinorelbine, vindesine, vincristine, vinblastine, vinflunine, ABT-751, verubulin, lexibulin, denibulin, indibulin, combrestatin A4, combrestatin A1, AVE8062 or a pharmaceutically acceptable salt thereof for the treatment of said tumor disease, particularly breast cancer.

In a preferred embodiment, a patient having a tumor disease, particularly breast cancer, may be separately, simultaneously or sequentially administered (a) a phosphatidylinositol 3-kinase (PI3K) inhibitor selected from COMPOUND A, COMPOUND B or a pharmaceutically acceptable salts thereof, and (b) a microtubule destabilizing agent eribulin or a pharmaceutically acceptable salt thereof for the treatment of said tumor disease, particularly breast cancer.

The combination disclosed herein may be particularly useful for the treatment of various tumor diseases mediated by, especially dependent on, the activity of PI3K (particularly the alpha-subunit of PI3K). Such tumor diseases mediated by the activity of PI3K may include, but are not limited to, those showing amplification of PI3K alpha, somatic mutation of PIK3CA or mutations and translocation of p85α that serve to up-regulate the p85-p110 complex. In one embodiment, the tumor diseases treated with the combination of the present invention is a cancer having amplification of PI3K alpha or somatic mutation of PIK3CA. In a further embodiment, the tumor diseases treated with the combination of the present invention is a breast cancer having amplification of PI3K alpha or somatic mutation of PIK3CA.

The administration of a COMBINATION OF THE INVENTION may result not only in a beneficial effect, e.g. therapeutic effect as compared to monotherapy of (a) phosphatidylinositol 3-kinase (PI3K) inhibitor selected from COMPOUND A, COMPOUND B or a pharmaceutically acceptable salts thereof and (b) a microtubule destabilizing agent, e.g, a synergistic therapeutic effect, e.g. with regard to alleviating, delaying progression of or inhibiting the symptoms, but also in further surprising beneficial effects, e.g. fewer side-effects, e.g. an improved quality of life or e.g. a decreased morbidity, compared with a monotherapy applying only one of the pharmaceutically active ingredients used in the combination of the invention.

A further benefit is that lower doses of the active ingredients of the COMBINATION OF THE INVENTION can be used, for example, that the dosages need not only often be smaller, but are also applied less frequently, or can be used in order to diminish the incidence of side-effects observed with one of the combination partners alone. This is in accordance with the desires and requirements of the patients to be treated.

It can be shown by established test models that a COMBINATION OF THE INVENTION results in the beneficial effects described herein before. The person skilled in the art is fully enabled to select a relevant test model to prove such beneficial effects. The pharmacological activity of a COMBINATION OF THE INVENTION may, for example, be demonstrated in a clinical study or in an in vivo or in vitro test procedure as essentially described hereinafter.

Suitable clinical studies are in particular, for example, open label, dose escalation studies in patients with a tumor disease, particularly breast cancer (including but not limited to breast cancer resistant to at least one prior chemotherapeutic regimen). Such studies prove in particular the synergism of the therapeutic agents of the combination of the invention. The beneficial effects on tumor diseases may be determined directly through the results of these studies which are known as such to a person skilled in the art. Such studies may be, in particular, be suitable to compare the effects of a monotherapy using either therapeutic agent and a combination of the invention. In one embodiment, the dose of the phosphatidylinositol 3-kinase inhibitor selected from COMPOUND A, COMPOUND B, or a pharmaceutically acceptable salt thereof, is escalated until the Maximum Tolerated Dosage is reached, and the microtubule destabilizing agent, e.g, eribulin or a pharmaceutically acceptable salt thereof, is administered with a fixed dose. Alternatively, phosphatidylinositol 3-kinase inhibitor selected COMPOUND A, COMPOUND B, or a pharmaceutically acceptable salt thereof, may be administered in a fixed dose and the dose of the microtubule destabilizing agent, e.g, eribulin or a pharmaceutically acceptable salt thereof, may be escalated. Each patient may receive doses of the phosphatidylinositol 3-kinase inhibitor either daily or intermittently. The efficacy of the treatment may be determined in such studies, e.g., after 12, 18 or 24 weeks by evaluation of symptom scores every 6 weeks.

In one aspect, the invention provides a pharmaceutical composition comprising a quantity, which is jointly therapeutically effective against a tumor disease, of the COMBINATION OF THE INVENTION. In this composition, the combination partners (a) and (b) are administered in a single formulation or unit dosage form by any suitable route. The unit dosage form may also be a fixed combination.

In a further aspect, the invention provides pharmaceutical compositions separately comprising a quantity, which is jointly therapeutically effective against a tumor disease, of combination partner (a) and combination partner (b) which are administered concurrently but separately, or administered sequentially.

The pharmaceutical compositions for separate administration of the combination partners, or for the administration in a fixed combination, i.e. a single galenical composition comprising the COMBINATION OF THE INVENTION, may be prepared in a manner known per se and are those suitable for enteral (such as oral or rectal) and parenteral administration to subjects and comprising a therapeutically effective amount of at least one combination partner alone, e.g. as indicated above, or in combination with one or more pharmaceutically acceptable carriers.

Preferably, the pharmaceutical composition comprising the PI3K inhibitor COMPOUND A, COMPOUND B, or any pharmaceutically acceptable salt thereof is suitable for enteral administration.

Preferably, the pharmaceutical composition comprising the microtubule destabiling agent eribulin or its pharmaceutically acceptable salt, particularly its mesylate salt form, is suitable for parenteral administration.

The novel pharmaceutical composition contains may contain, from about 0.1% to about 99.9%, preferably from about 1% to about 60%, of the active ingredient(s).

Pharmaceutical compositions for the combination therapy, including fixed combinations or non-fixed combinations, for enteral or parenteral administration are, for example, those in unit dosage forms, such as sugar-coated tablets, tablets, capsules or suppositories, or ampoules. If not indicated otherwise, these are prepared in a manner known per se, for example by means of various conventional mixing, comminution, granulating, sugar-coating, dissolving, lyophilizing processes, or fabrication techniques readily apparent to those skilled in the art. It will be appreciated that the unit content of a combination partner contained in an individual dose of each dosage form need not in itself constitute an effective amount since the necessary effective amount may be reached by administration of a plurality of dosage units. It will be further appreciated that the unit content of a combination partner for parenteral administration may contain a higher dosage amount of the combination partner which is diluted to the effective dosage amount before administration.

A unit dosage form containing the combination of agents or individual agents of the combination of agents may be in the form of micro-tablets enclosed inside a capsule, e.g. a gelatin capsule. For this, a gelatin capsule as is employed in pharmaceutical formulations can be used, such as the hard gelatin capsule known as CAPSUGEL™, available from Pfizer.

The unit dosage forms of the present invention may optionally further comprise additional conventional carriers or excipients used for pharmaceuticals. Examples of such carriers include, but are not limited to, disintegrants, binders, lubricants, glidants, stabilizers, and fillers, diluents, colorants, flavours and preservatives. One of ordinary skill in the art may select one or more of the aforementioned carriers with respect to the particular desired properties of the dosage form by routine experimentation and without any undue burden. The amount of each carriers used may vary within ranges conventional in the art. The following references which are all hereby incorporated by reference disclose techniques and excipients used to formulate oral dosage forms. See The Handbook of Pharmaceutical Excipients, 4^{th} edition, Rowe et al., Eds., American Pharmaceuticals Association (2003); and Remington: the Science and Practice of Pharmacy, 20^{th} edition, Gennaro, Ed., Lippincott Williams & Wilkins (2003).

These optional additional conventional carriers may be incorporated into the oral dosage form either by incorporating the one or more conventional carriers into the initial mixture before or during melt granulation or by combining the one or more conventional carriers with the granules in the oral dosage form. In the latter embodiment, the combined mixture may be further blended, e.g., through a V-blender, and subsequently compressed or molded into a tablet, for example a monolithic tablet, encapsulated by a capsule, or filled into a sachet.

Examples of pharmaceutically acceptable disintegrants include, but are not limited to, starches; clays; celluloses; alginates; gums; cross-linked polymers, e.g., cross-linked polyvinyl pyrrolidone or crospovidone, e.g., POLYPLASDONE XL™ from International Specialty Products (Wayne, N.J.); cross-linked sodium carboxymethylcellulose or croscarmellose sodium, e.g., AC-DI-SOL™ from FMC; and cross-linked calcium carboxymethylcellulose; soy polysaccharides; and guar gum. The disintegrant may be present in an amount from about 0% to about 10% by weight of the composition. In one embodiment, the disintegrant is present in an amount from about 0.1% to about 5% by weight of composition.

Examples of pharmaceutically acceptable binders include, but are not limited to, starches; celluloses and derivatives thereof, for example, microcrystalline cellulose, e.g., AVICEL PH™ from FMC (Philadelphia, Pa.), hydroxypropyl cellulose hydroxylethyl cellulose and hydroxylpropylmethyl cellulose METHOCEL™ from Dow Chemical Corp. (Midland, Mich.); sucrose; dextrose; corn syrup; polysaccharides; and gelatin. The binder may be present in an amount from about 0% to about 50%, e.g., 2-20% by weight of the composition.

Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. The lubricant may be present in an amount from about 0% to about 10% by weight of the composition. In one embodiment, the lubricant may be present in an amount from about 0.1% to about 1.5% by weight of composition. The glidant may be present in an amount from about 0.1% to about 10% by weight.

Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 0% to about 80% by weight of the composition.

The optimum ratios, individual and combined dosages, and concentrations of the therapeutic agents of the COMBINATON OF THE INVENTION that yield efficacy without toxicity are based on the kinetics of the therapeutic agent's availability to target sites, and are determined using methods known to those of skill in the art.

In one embodiment, the combination partners (a) and (b) of the present invention (for example, the phosphatidylinositol 3-kinase inhibitor selected COMPOUND A, COMPOUND B, or a pharmaceutically acceptable salt thereof and a microtubule destabilizing agent, e.g., eribulin or a pharmaceutically acceptable salt thereof) may be present in the combinations, pharmaceutical compositions and dosage forms disclosed herein in a ratio in the range of 1:1 to 500:1, e.g, 400:1, 300:1, 275:1, 100:1, or 10:1.

In accordance with the present invention, a therapeutically effective amount of each of the therapeutic agents of the COMBINATON OF THE INVENTION may be administered simultaneously or sequentially and in any order, and the components may be administered separately or as a fixed combination. For example, the method of treating a tumor disease according to the invention may comprise (i) administration of the first agent (a) in free or pharmaceutically acceptable salt form, and (ii) administration of an agent (b) in free or pharmaceutically acceptable salt form, simultaneously or sequentially in any order, in jointly therapeutically effective amounts, preferably in synergistically effective amounts, e.g. in daily or intermittently dosages corresponding to the amounts described herein. The individual therapeutic agents of the COMBINATION OF THE INVENTION may be administered separately at different times during the course of therapy or concurrently in divided or single combination forms. Furthermore, the term “administering” also encompasses the use of a pro-drug of a therapeutic agent that convert in vivo to the therapeutic agent as such. The instant invention is therefore to be understood as embracing all such regimens of simultaneous or alternating treatment and the term “administering” is to be interpreted accordingly.

The effective dosage of each of the therapeutic agents employed in the COMBINATION OF THE INVENTION may vary depending on the particular therapeutic agent or pharmaceutical composition employed, the mode of administration, the condition being treated, and the severity of the condition being treated. Thus, the dosage regimen of the COMBINATION OF THE INVENTION is selected in accordance with a variety of factors including the route of administration and the renal and hepatic function of the patient. A clinician or physician of ordinary skill can readily determine and prescribe the effective amount of the single active ingredients required to alleviate, counter or arrest the progress of the condition.

The effective dosage of each of the therapeutic agents of the COMBINATION OF THE INVENTION may require more frequent administration of one of the therapeutic agent(s) as compared to the other therapeutic agent(s) in the combination. Therefore, to permit appropriate dosing, packaged pharmaceutical products may contain one or more dosage forms that contain the combination of compounds, and one or more dosage forms that contain one of the combination of therapeutic agent(s), but not the other therapeutic agent(s) of the combination.

When the combination partners, which are employed in the COMBINATION OF THE INVENTION, are applied in the form as marketed as single drugs, their dosage and mode of administration can be in accordance with the information provided on the package insert of the respective marketed drug, if not mentioned herein otherwise.

COMPOUND A, is preferably administered daily at a dose in the range of from about 1.0 to 30 mg/kg body weight. In one preferred embodiment, the dosage of COMPOUND A, is in the range of about 10 mg to about 200 mg/day, preferably from about 50 mg to about 200 mg or more preferably from about 60 mg to about 120 mg or most preferably about 100 mg per day, especially if the subject is an adult human.

COMPOUND B, is preferably administered daily at a dose in the range of from about 0.05 to about 50 mg per kilogram body weight of the recipient per day; preferably about 0.1-25 mg/kg/day, more preferably from about 0.5 to 10 mg/kg/day. Thus, for administration to a 70 kg person, the dosage range may be about 35-700 mg per day, preferably 200-500 mg or 300-400 mg.

Eribulin, especially its mesylate salt form, is preferably administered intravenously at a dose in the range from about 0.7 mg/m² to 1.5 mg/m² over 2 to 5 minutes on Days 1 and 8 of a 21-day cycle. In one preferred embodiment, the dosage of eribulin, especially its mesylate salt form, is administered intravenously at a dosage of 1.4 mg/m² over 2 to 5 minutes on Days 1 and 8 of a 21-day cycle to an adult person.

Vinorelbine, particularly its tartrate salt, is preferably administered intravenously at a dose in the range from about 7 mg/m² to about 35 mg/m² over 6 to 10 minutes and weekly. In a preferred embodiment, the dosage of vinorelbine, particularly its tartrate salt, is administered intraveneously at a dosage of 30 mg/m² over 6 to 10 minutes and weekly to an adult person.

The optimal dosage of each therapeutic agent for treatment of a tumor disease can be determined empirically for each individual using known methods and will depend upon a variety of factors, including, though not limited to, the degree of advancement of the disease; the age, body weight, general health, gender and diet of the individual; the time and route of administration; and other medications the individual is taking. Optimal dosages may be established using routine testing and procedures that are well known in the art.

The amount of each therapeutic agent that may be combined with the carrier materials to produce a single dosage form will vary depending upon the individual treated and the particular mode of administration. In some embodiments the unit dosage forms containing the combination of therapeutic agents as described herein will contain the amounts of each agent of the combination that are typically administered when the therapeutic agents are administered alone.

Frequency of dosage may vary depending on the therapeutic agent used and the particular condition to be treated. In general, the use of the minimum dosage that is sufficient to provide effective therapy is preferred. Patients may generally be monitored for therapeutic effectiveness using assays suitable for the condition being treated, which will be familiar to those of ordinary skill in the art.

In one aspect, the present invention provides a method of treating a tumor disease comprising administering to subject in need thereof a COMBINATION OF THE INVENTION in a quantity, which is jointly therapeutically effective against said tumor disease. In one embodiment, the tumor disease to be treated with a COMBINATION OF THE INVENTION is breast cancer, preferably breast cancer resistant to at least one prior chemotherapeutic regimen.

Moreover, the present invention also provides a method of inhibiting the formation of metastases in a subject having tumor disease, in particular a breast cancer, comprising administering to a subject in need thereof an amount of a COMBINATION OF THE INVENTION in a quantity which is therapeutically effective against said tumor disease.

In one aspect, the present invention provides the use of a COMBINATION OF THE INVENTION for the treatment of a tumor disease, in particular breast cancer, and/or for the preparation of a medicament for the treatment of a tumor disease.

In one aspect, the present invention provides the use of a COMBINATION OF THE INVENTION for the inhibition of the formation of metastases in a subject having a tumor disease, particularly breast cancer, and/or for the preparation of a medicament for the inhibition of the formation of metastases in a subject having a tumor disease.

In one aspect, the present invention provides a commercial package comprising as active ingredients COMBINATION OF THE INVENTION, together with instructions for the simultaneous, separate or sequential use thereof in the treatment of a tumor disease, particularly breast cancer.

In one aspect, the present invention provides a commercial package comprising a phosphatidylinositol 3-kinase (PI3K) inhibitor selected from 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salts thereof and instructions for the simultaneous, separate or sequential use with a microtubule destabilizing agent selected from eribulin, vinorelbine, vindesine, vincristine, vinblastine, vinflunine, ABT-751, verubulin, lexibulin, denibulin, indibulin, combrestatin A4, combrestatin A1, AVE8062 or a pharmaceutically acceptable salt thereof, in the treatment of a tumor disease.

The following Examples illustrate the invention described above; they are not, however, intended to limit the scope of the invention in any way. The beneficial effects of the pharmaceutical combination of the present invention can also be determined by other test models known as such to the person skilled in the pertinent art.

Utility of the COMBINATION OF THE PRESENT INVENTION, as described herein, may be demonstrated in vitro, in animal test methods as well as in clinical studies. For example in the utility of the compounds of formula (I) in accordance with the present invention may be demonstrated in accordance with the methods hereinafter described:

### Example 1

In experiments, Annexin V staining was used to determine the quantity of apoptotic HCC1143 triple-negative breast cancer cells (PTEN normal) and/or MDA-MB-468 triple-negative breast cancer cells (characterized with PTEN loss) after treatment with either the combination of COMPOUND A hydrochloride salt with eribulin mesylate, control, COMPOUND A hydrochloride monotherapy or eribulin mesylate monotherapy. Data shows the results for treatments at EC50 and 48 hours.

Eribulin mesylate may be obtained under the tradename HALAVEN® (Eisai) 0.44 mg/mL solution for injection. One mL solution contains 0.44 mg of eribulin (equivalent to 0.5 mg eribulin mesylate).

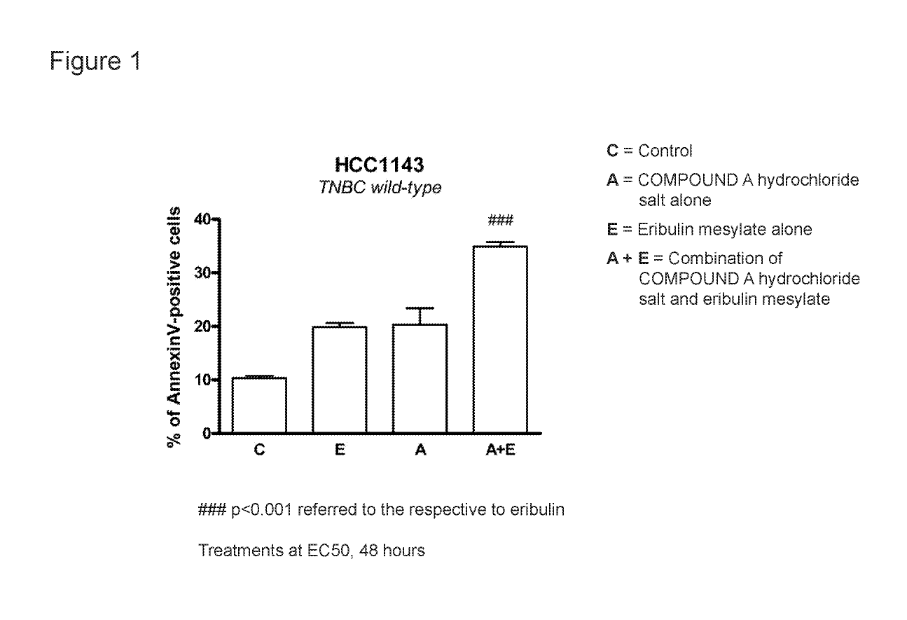

As shown in FIG. 1, the combination of COMPOUND A hydrochloride salt and eribulin mesylate enhanced the annexin V-positive (apoptotic) HCC1143 triple negative breast cancer cells as compared to those cells treated with control, COMPOUND A hydrochloride monotherapy or eribulin mesylate monotherapy. The combination of COMPOUND A hydrochloride salt with eribulin mesylate resulted in a statistically significant higher percentage of annexin V-positive (apoptotic) HCC11433 triple negative breast cancer cells as compared to eribulin mesylate monotherapy.

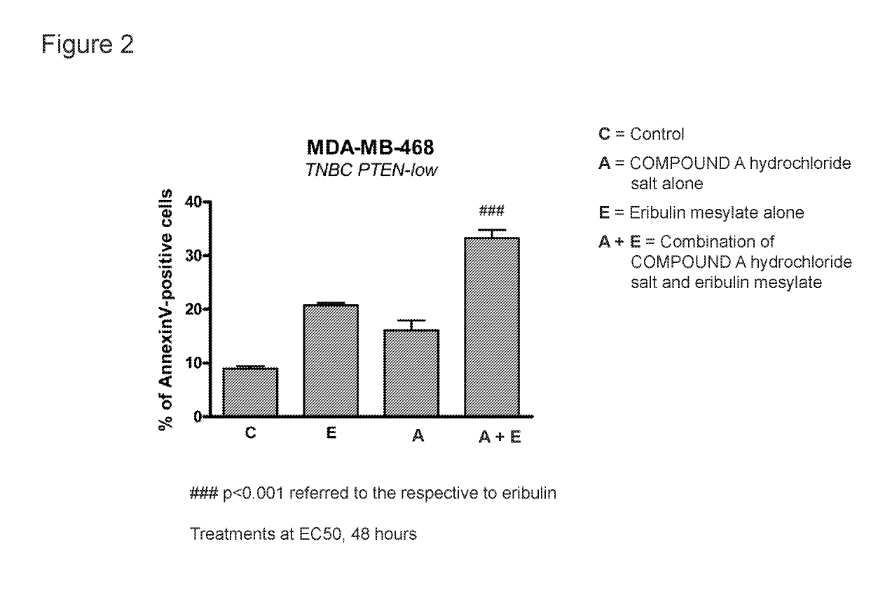

As shown in FIG. 2, the combination of COMPOUND A hydrochloride salt and eribulin mesylate enhanced the annexin V-positive (apoptotic) MDA-MB-468 triple-negative breast cancer cells as compared to those cells treated with control, COMPOUND A hydrochloride monotherapy or eribulin mesylate monotherapy. The combination of COMPOUND A hydrochloride salt with eribulin mesylate resulted in a statistically significant higher percentage of annexin V-positive (apoptotic) MDA-MB-468 triple-negative breast cancer cells as compared to eribulin mesylate monotherapy.

### Example 2

Experiments were conducted to evaluate the response of triple negative breast cancer tumors to treatment with either (a) combination of COMPOUND A hydrochloride salt with eribulin mesylate, (b) COMPOUND A hydrochloride salt monotherapy, (c) eribulin mesylate monotherapy, and (d) control.

Xenografts were developed by subcutaneously implanting MDA-MB-468 triple-negative breast cancer cells (characterized with PTEN loss) into female mice.

Tumor-bearing mice were administered either: (a) control, (b) COMPOUND A hydrochloride salt alone, (c) eribulin mesylate alone, or (d) COMPOUND A hydrochloride salt with eribulin mesylate. Tumor-bearing mice receiving treatment of (b) COMPOUND A hydrochloride salt alone, (c) eribulin mesylate alone, or (d) COMPOUND A hydrochloride salt with eribulin mesylate. COMPOUND A hydrochloride salt was administered in a clinically equivalent dose of 27.5 mg/kg, and eribulin mesylate was administered in a clinically equivalent dose of 0.1 mg/kg. Tumor volumes were measured and recorded regularly during treatment.

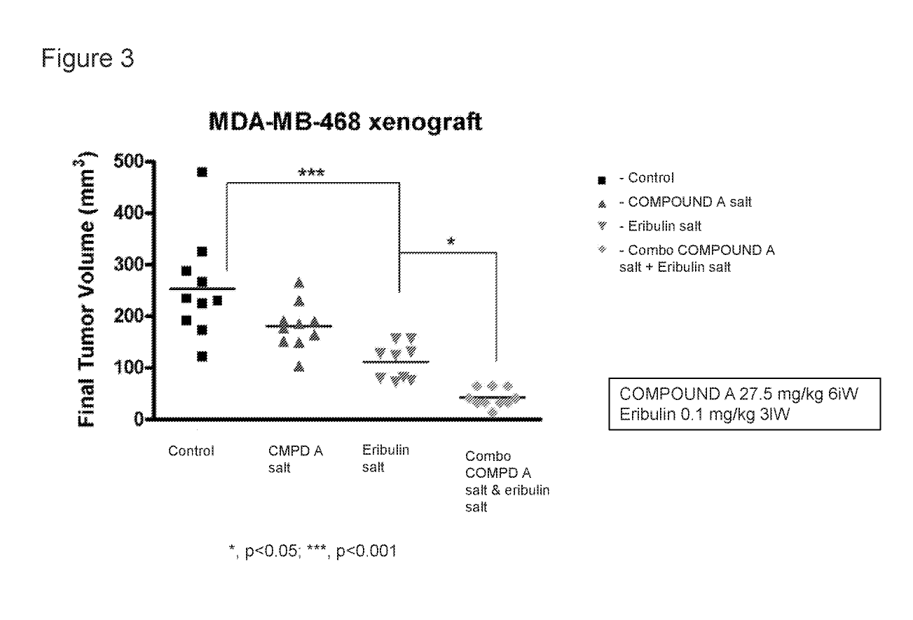

FIG. 3 shows the final tumor volumes (mm³) for those MDA-MB-468 triple-negative breast cancer cell tumor-bearing mice treated with the combination of COMPOUND A hydrochloride salt with eribulin mesylate as compared to control, COMPOUND A hydrochloride salt monotherapy and eribulin mesylate monotherapy.

### Example 3

Experiments were conducted to evaluate the response of triple negative breast cancer tumors to treatment with either (a) combination of COMPOUND A hydrochloride salt with eribulin mesylate, (b) COMPOUND A hydrochloride salt monotherapy, (c) eribulin mesylate monotherapy, and (d) control.

Patient-derived xenografts (PDXs) from metastatic breast cancer patients were developed by subcutaneously implanting the tumor into female mice. In this experiment, a triple-negative breast cancer tumor (PDX44) characterized with a PIK3CA H1047R mutation and PTEN loss was subcutaneously implanted into the female mice.

Tumor-bearing mice were administered either: (a) control, (b) COMPOUND A hydrochloride salt alone, (c) eribulin mesylate alone, or (d) COMPOUND A hydrochloride salt with eribulin mesylate. Tumor-bearing mice receiving treatment of (b) COMPOUND A hydrochloride salt alone, (c) eribulin mesylate alone, or (d) COMPOUND A hydrochloride salt with eribulin mesylate were treated for 42 days. COMPOUND A hydrochloride salt was administered in a clinically equivalent dose of 27.5 mg/kg, and eribulin mesylate was administered in a clinically equivalent dose of 0.1 mg/kg. Tumor volumes were measured and recorded regularly during treatment. Tumor bearing mice treated with control were discontinued from the experiment after 24 days due to tumor progression.

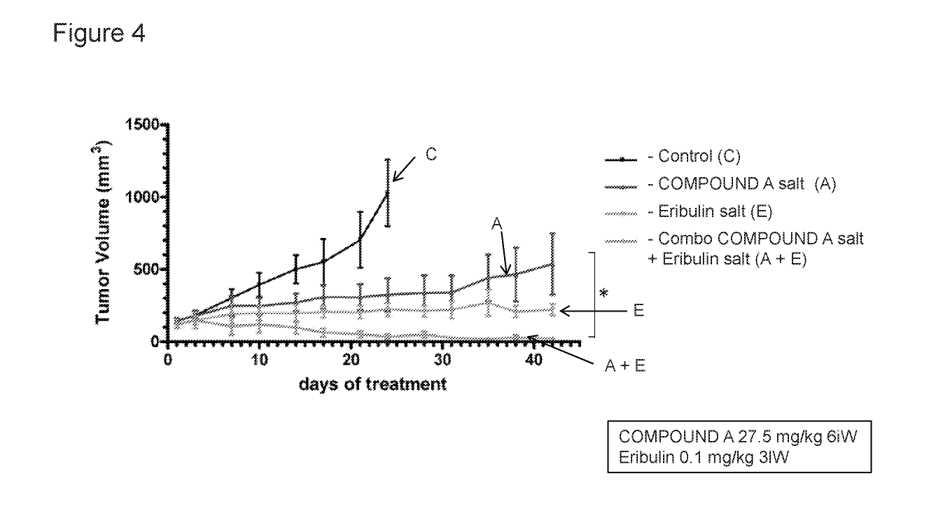

FIG. 4 shows the improved antitumor activity of the combination of COMPOUND A hydrochloride salt with eribulin mesylate in this triple negative breast cancer patient-derived xenograft model as compared to control, COMPOUND A hydrochloride salt monotherapy and eribulin mesylate monotherapy. The mice treated with the combination of COMPOUND A hydrochloride salt and eribulin mesylate demonstrated a statistically significant reduced tumor volume as compared to COMPOUND A hydrochloride salt monotherapy and eribulin mesylate monotherapy at 42 days treatment.

### Example 4

Experiments were conducted to evaluate the response of triple negative breast cancer tumors to treatment with either (a) combination of COMPOUND A hydrochloride salt with eribulin mesylate, (b) COMPOUND A hydrochloride salt monotherapy, (c) eribulin mesylate monotherapy, and (d) control.

Xenografts were developed by subcutaneously implanting CAL51 triple-negative breast cancer cells (characterized with a PIK3CA E542K mutation and PTEN loss) into female mice.

Tumor-bearing mice were administered either: (a) control, (b) COMPOUND A hydrochloride salt alone, (c) eribulin mesylate alone, or (d) COMPOUND A hydrochloride salt with eribulin mesylate. Tumor-bearing mice receiving treatment of (b) COMPOUND A hydrochloride salt alone, (c) eribulin mesylate alone, or (d) COMPOUND A hydrochloride salt with eribulin mesylate were treated for 46 days. COMPOUND A hydrochloride salt was administered in a clinically equivalent dose of 27.5 mg/kg, and eribulin mesylate was administered in a clinically equivalent dose of 0.1 mg/kg. Tumor volumes were measured and recorded regularly during treatment. Tumor bearing mice treated with control were discontinued from the experiment after 29 days due to tumor progression.

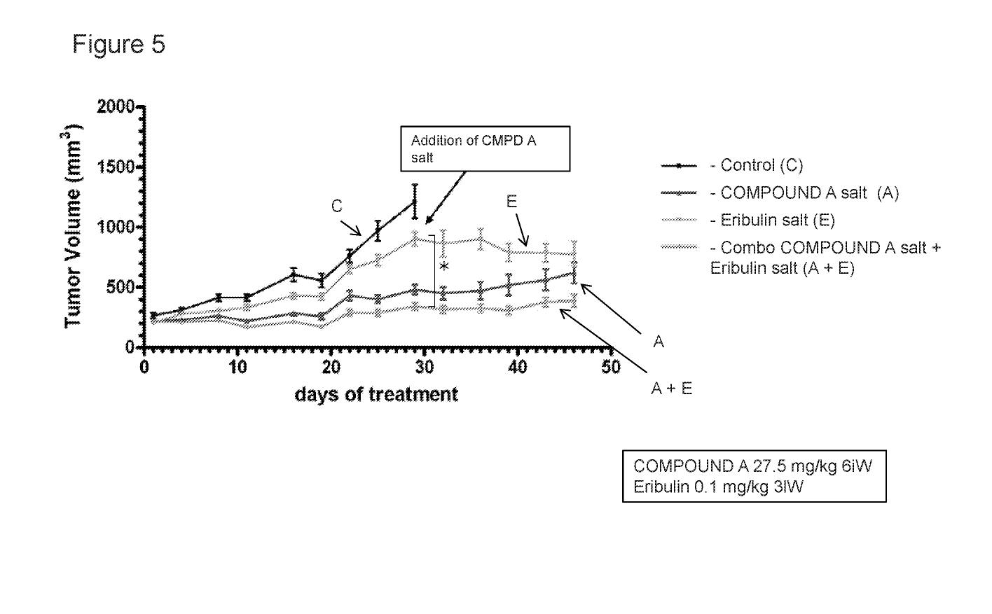

FIG. 5 shows the improved antitumor activity of the combination of COMPOUND A hydrochloride salt with eribulin mesylate in this triple negative breast cancer xenograft model as compared to control, COMPOUND A hydrochloride salt monotherapy and eribulin mesylate monotherapy. At 29 days of treatment, the mice treated with the combination of COMPOUND A hydrochloride salt and eribulin mesylate demonstrated a statistically significant reduced tumor volume as compared to COMPOUND A hydrochloride salt monotherapy and eribulin mesylate monotherapy.

After 29 days, those mice treated with eribulin mesylate alone were additionally administered COMPOUND A hydrochloride salt. FIG. 5 shows the comparative change of tumor volume for these mice during treatment with eribulin mesylate alone and during treatment with the combination.

### Example 5

Experiments were conducted to evaluate the response of triple negative breast cancer tumors to treatment with either (a) combination of COMPOUND B with eribulin mesylate, (b) COMPOUND B monotherapy, (c) eribulin mesylate monotherapy, and (d) control.

Patient-derived xenografts (PDXs) from metastatic breast cancer patients are developed by subcutaneously implanting the tumor into female mice. In this experiment, a triple-negative breast cancer tumor (PDX44) characterized with a PIK3CA H1047R mutation and PTEN loss is subcutaneously implanted into these female mice.

Tumor-bearing mice are administered either: (a) control, (b) COMPOUND B alone, (c) eribulin mesylate alone, or (d) COMPOUND B with eribulin mesylate for 25-30 days. Tumor volumes are measured and recorded regularly during treatment.

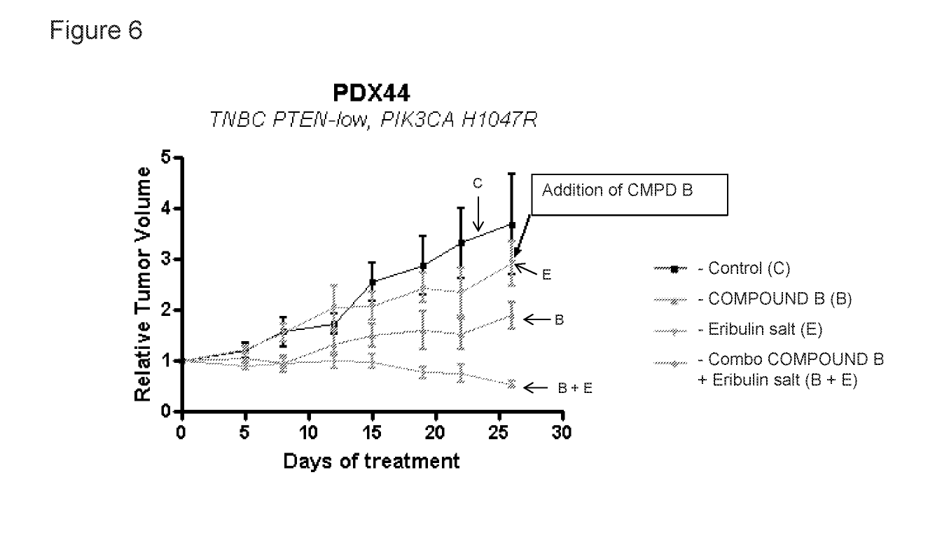

FIG. 6 shows the improved antitumor activity of the combination of COMPOUND B with eribulin mesylate in this PDX44 triple negative breast cancer patient-derived xenograft model as compared to control, COMPOUND B monotherapy and eribulin mesylate monotherapy. The mice treated with the combination of COMPOUND B and eribulin mesylate resulted in a reduction of tumor volume as compared to the starting tumor volume. Further, the mice treated with this combination demonstrated a reduced tumor volume as compared to control, COMPOUND B monotherapy and eribulin mesylate monotherapy at 25 days treatment.

### Example 6

### Modulation of Eribulin Antitumor Activity by PI3K Blockade in PIK3CA-Mutant Eribulin-Resistant Tumor Xenografts

Eribulin is a recently approved microtubule destabilizing agent for the management of heavily pretreated HER2-negative metastatic breast cancer (BC) patients. We hypothesized that PI3K-pathway activation limits the antitumor activity of eribulin in HER2-negative BC and that PI3K inhibition enhances the efficacy of this microtubule destabilizing agent. The predictive value of PIK3CA mutation or PTEN loss towards eribulin response was interrogated using cell line- and patient-derived tumor models. While PIK3CA mutation seemed to be not predictive in vitro, the PIK3CA-mutated xenograft models underwent tumor progression upon single-agent eribulin therapy.

In experiments, Patient- (PDX) and cell line-derived tumor xenografts were treated with eribulin mesylate (0.1 mg/kg, 3IW) and/or COMPOUND A hydrochloride salt (27.5 mg/kg, 6IW). Table 1 summarizes the percentage change in tumor volume after 26-31 days of treatment:

TABLE 1

| Status | PIK3CA | PIK3CA mutant | | PTEN-low | | |
|---|---|---|---|---|---|---|
| Model/ | mutant | and PTEN-low | | MDA- | | WT |
| Treatment | MCF7L | PDX44 | CAL51 | MB-468 | PDX88 | PDX98 |
| Eribulin | 39 ± 96 | 83 ± 51 | 364 ± 127 | −47 ± 14 | −26 ± 34 | −36 ± 33 |
| COMPOUND A salt | 501 ± 169 | 116 ± 111 | 119 ± 73 | −9 ± 23 | 128 ± 37 | 87 ± 112 |
| Eribulin + | −70 ± 27 | −80 ± 10 | 51 ± 46 | −75 ± 12 | −94 ± 5 | −65 ± 13 |
| COMPOUND A salt | | | | | | |

PTEN loss was neither predictive in vitro nor in vivo. Moreover, eribulin induced PI3K-pathway activation in tumor xenografts and could therefore explain a mechanism by which some tumors escape to microtubule destabilizing agent-therapy.

Class I pan-PI3K (COMPOUND A hydrochloride salt) or PI3K-α-specific inhibitors (COMPOUND B) were used in vitro to block the PI3K-pathway concomitantly with eribulin treatment, resulting in enhanced antiproliferative and proapoptotic activity. Strikingly, in PIK3CA mutant xenograft models, eribulin did not exhibit antitumor activity, while co-administration of a PI3K inhibitor (COMPOUND A hydrochloride salt or COMPOUND B) induced marked tumor regression. Moreover, addition of the PI3K inhibitor at progression to eribulin single-agent equally resulted in tumor regression. Of note, PIK3CA wild-type models likewise exhibited an increased antitumor activity with the combined therapy compared to single-agent treatments. The precise mechanism by which the combination of eribulin and a PI3K-targeting agent results in tumor regression is currently under investigation.

These results support the clinical development of therapeutic regimens combining PI3K-inhibitors to the approved microtubule destabilizing agent eribulin and might be indicative of clinical benefit both in the PIK3CA mutant and wild type breast cancer population.

1. A combination comprising (a) a phosphatidylinositol 3-kinase (PI3K) inhibitor selected from 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salts thereof, and (b) a microtubule destabilizing agent selected from eribulin, vinorelbine, vindesine, vincristine, vinblastine, vinflunine, ABT-751, verubulin, lexibulin, denibulin, indibulin, combrestatin A4, combrestatin A1, AVE8062 or a pharmaceutically acceptable salt thereof, for simultaneous, separate or sequential use.

2. A combination according to claim 1, wherein the PI3K inhibitor is 5-(2,6-di-morpholin-4-yl-pyrimidin-4-yl)-4-trifluoromethyl-pyridin-2-ylamine or a pharmaceutically acceptable salt thereof.

3. A combination according to claim 1, wherein the PI3K inhibitor is (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salts thereof.

4. A combination according to claim 1, wherein the microtubule destabilizing agent is eribulin or a pharmaceutically acceptable salt thereof.

5. (canceled)

6. (canceled)

7. A pharmaceutical composition comprising a combination according to any one of claim 1 and at least one pharmaceutically acceptable carrier.

8. A method of treating a subject having a tumor disease which comprises administering to said subject a combination according to claim 1 in a quantity which is jointly therapeutically effective against said tumor disease.

9. A method of inhibiting the formation of metastases in a subject having a tumor disease which comprises administering to a subject in need thereof a pharmaceutically effective amount of a combination according to claim 1.

10. A method according to claim 8 or 9, wherein the microtubule destabilizing agent is eribulin mesylate.

11. A method according to claim 8 or 9, wherein the tumor disease is breast cancer.

12-15. (canceled)

16. A commercial package comprising a phosphatidylinositol 3-kinase (PI3K) inhibitor selected from 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salts thereof and instructions for the simultaneous, separate or sequential use with a microtubule destabilizing agent selected from eribulin, vinorelbine, vindesine, vincristine, vinblastine, vinflunine, ABT-751, verubulin, lexibulin, denibulin, indibulin, combrestatin A4, combrestatin A1, AVE8062 or a pharmaceutically acceptable salt thereof, in the treatment of a tumor disease.

17. A method according to claim 8, wherein the PI3K inhibitor is 5-(2,6-di-morpholin-4-yl-pyrimidin-4-yl)-4-trifluoromethyl-pyridin-2-ylamine or a pharmaceutically acceptable salt thereof.

18. A method according to claim 8, wherein the PI3K inhibitor is (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salts thereof.

19. A method according to claim 8, wherein the tumor disease is selected from benign or malignant tumors, brain cancer, kidney cancer, liver cancer, bladder cancer, breast cancer, gastric cancer, ovarian cancer, colon cancer, rectum cancer, prostate cancer, pancreatic cancer, lung cancer (including non-small cell lung cancer and small cell lung cancer), bronchial cancer, vaginal cancer, hepatocellular carcinoma, intrahepatic bile duct cancer, glioma, glioblastoma, cervical cancer, bladder cancer, esophageal cancer, cancer of the head and neck, thyroid cancer, melanoma, endometrial cancer, multiple myeloma, acute myelogeous leukemia, chronic myelogenous leukemia, lymphocytic leukemia, non-Hodgkin lymphoma, gastrointestinal cancer, or any combination thereof.
